(19) United States
(12) Patent Application Publication
(10) Pub. No.: US 2002/0177876 A1
(43) Pub. Date: Nov. 28, 2002

(54) Polyolefin sutures having improved processing and handling characteristics

(75) Inventors: Mark S. Roby, Killingworth, CT (US); John J. Kennedy, Guilford, CT (US); Richard Stevenson, Colchester, CT (US)

Correspondence Address: Mark Farber, c/o Tyco Healthcare Group LP, 150 Glover Avenue, Norwalk, CT, 06856, US

(73) Assignee: Tyco Healthcare Group LP

(21) Appl. No.: 10/103,187

(22) Filed: Mar. 20, 2002

Related U.S. Application Data
(60) Provisional application No. 60/278,686, filed on Mar. 26, 2001.

(51) Int. Cl. A61B 17/04

(52) U.S. Cl. 606/228

(57) ABSTRACT

A suture filament is made from a polyolefin such as polypropylene which contains a fatty acid diester of polyethylene glycol, such as, for example, polyethylene glycol distearate.

### BACKGROUND

1. Technical Field

The present disclosure relates to surgical sutures, and particularly to a polypropylene surgical suture having improved processing and handling characteristics.

2. Background of the Related Art

Polyolefin sutures are known in the art. Such sutures are non-absorbable and generally include polypropylene or polymeric combinations of ethylene and propylene. The polymeric components of the polyolefin sutures are generally melt spun to produce filaments for use in fabricating the surgical suture strands. Polypropylene sutures are advantageously produced as monofilament sutures.

Various methods are known for making polypropylene sutures. For example, U.S. Pat. No. 5,217,485 to Liu et al. discloses a process for making a polypropylene monofilament suture by melt extruding the monofilament, stretching the solidified monofilament, then allowing the monofilament to equilibrate, or “rest”, prior to annealing.

Polypropylene monofilament sutures are known to exhibit a limited amount of fraying as the suture passes over itself, e.g., when tying knots. While the limited amount of fraying exhibited by polypropylene monofilament sutures does not substantially hamper the performance of the suture, there remains room for improvements to be made in the processing and handling characteristics of such sutures.

### SUMMARY

It has now been found that the processing and handling characteristics of polyolefin sutures can be improved by incorporating a fatty acid diester of polyethylene glycol into the polyolefin resin prior to spinning of the filament(s). A method for fabricating a polyolefin suture is also provided herein. In the novel method described herein, a polyolefin is combined with an effective fray reducing amount of a fatty acid diester of polyethylene glycol, preferably polyethylene glycol distearate. The mixture of polyolefin and diester is heated to form a melt. The melt is then extruded to form a filament. The polyolefin is preferably polypropylene.

### BRIEF DESCRIPTION OF THE DRAWING(S)

FIG. 1 is a schematic illustration of apparatus which is suitable for carrying out the suture manufacturing process described herein; and

FIG. 2 is a depiction of a needled suture in accordance with the present disclosure.

### DETAILED DESCRIPTION OF PREFERRED EMBODIMENT(S)

All composition percentages listed herein shall be understood to be by weight unless otherwise indicated. All quantities set forth below, except in the claims, shall be understood to be modified by the term “about”.

The present disclosure relates to a composition from which filaments for sutures can be produced by melt extrusion, or “spinning”, of polyolefins. The preferred polyolefins include polyethylene, polypropylene, copolymers of polyethylene and polypropylene, and blends of polyethylene and polypropylene. Polypropylene is most preferred. The polypropylene can be isotactic polypropylene or a mixture of isotactic and syndiotactic or atactic polypropylene. Useful isotactic polypropylene resins include those described in U.S. Pat. No. 3,630,205 which is herein incorporated by reference, i.e., those possessing a weight average molecular weight (Mw) of from 294,000 to 316,000, a number average molecular weight (Mn) of 78,400 to 82,100 and a calculated dispersity (Mn/Mw) of from 3.58 to 4.0. Useful polypropylene resins will advantageously possess a melt flow index in g/10 min of 2 to 6 and preferably from 3.5 to 4.5. Isotactic polypropylene resins which can be used herein include Resin F040A Blue of Aristech Chemical Corporation (Pittsburgh, Pa.) and Profax 6523 of Himont Incorporated (Wilmington, Del.).

The composition includes a fatty acid diester to reduce fraying and facilitate suture formation. The fatty acid diester is preferably a diester of a polyalkylene glycol. Suitable fatty acids include C₁₀-C₂₆ fatty acids such as stearic, lauric, palmitic, myristic, arachidic, behenic, and similar acids. Suitable polyalkylene glycols include C₂-C₆ alklyene glycols, preferably polyethylene and polypropylene glycols.

In a first step for making a suture filament the polyolefin is combined with the fatty acid diester. The preferred fatty acid diester of polyethylene glycol such as, for example, polyethylene glycol distearate (PEG distearate). In particular, the preferred PEG distearate for use in the method described herein has a melting point of from about 35° C. to about 37° C., an acid value of about 5.0, an iodine value of 0.41, and a saponification value of about 117.0. A suitable PEG distearate is available from the Aldrich Chemical Co. of Milwaukee, Wis.

The composition percentage of the fatty acid diester in the final product can range from 0.01% to 1.0%, preferably 0.1% to 0.5%, most preferably 0.2% to 0.4%.

The first step of the method can be performed by directly adding fatty acid diester to the polypropylene (or other polyolefin) either prior to or during melting. Preferably, however, a mixture of polypropylene and fatty acid diester is prepared by making a master batch of preblended polypropylene containing polypropylene and fatty acid diester in a weight ratio of from 2:1 to 50:1. Then the master batch is mixed with a batch of standard polypropylene pellets to provide the overall desired level of fatty acid distearate. The weight ratio of standard polypropylene pellets to the master batch of preblended polypropylene (in pellet or other suitable form) containing fatty acid diester is from about 2:1 to 50:1. As those skilled in the art will appreciate, the ratio of standard polypropylene to the preblended polypropylene can be adjusted to produce a product having any target percentage composition of fatty acid diester. Mixing a small quantity of pre-blended polypropylene with standard polypropylene pellets achieves better dispersion of the fatty acid diester in the subsequent polymer melt than direct addition of diester to the polypropylene. The preblended polypropylene can be produced at one facility or operation and formed into a master batch of pellets which can then be stored and/or transferred to the suture fabrication operation. The polypropylene used to make the pre-blended batch of polypropylene/fatty acid diester preferably has the same characteristics (e.g., molecular weight, melt flow index, etc.) as the standard polypropylene with which the pre-blended batch is combined.

The next step in the method is heating the combined polyolefin and diester to form a polymer melt. This melt is then extruded and cooled to form a filament which can then be sent to further processing such as stretching. The melt contains substantially no water or organic solvents, and no substances which would be incompatible with body tissue. The polypropylene may contain some colorant to facilitate visualizing the suture filament during a surgical procedure.

Methods for extruding and processing filaments of polypropylene and other polyolefins are known in the art.

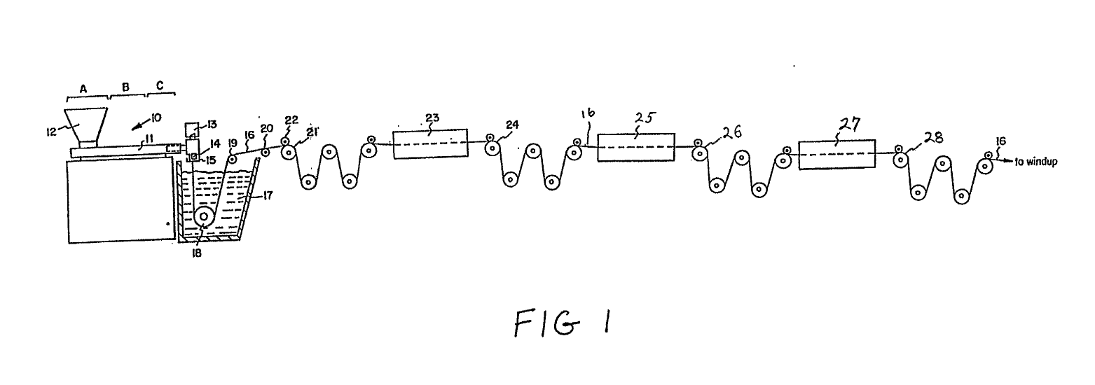

An exemplary process for manufacturing a suture is shown in FIG. 1, which schematically illustrates the extrusion and stretching operations of the polypropylene monofilament manufacturing operation herein. Extruder unit 10 is of a known or conventional type and is equipped with controls for regulating the temperature of barrel 11 in various zones thereof, e.g., progressively higher temperatures in three consecutive zones A, B and C along the length of the barrel. Pellets or powder of polypropylene resin, which have been mixed with pellets or powder of preblended polypropylene/fatty acid diester in the proportions indicated above, are introduced to the extruder through drier-hopper 12.

Motor-driven metering pump 13 delivers extruded resin at a constant rate to spin pack 14 and thereafter through spinneret 15 possessing one or more orifices of desired diameter to provide a molten monofilament 16 which then enters quench bath 17, e.g., containing water, where the monofilament solidifies. The distance monofilament 16 travels after emerging from spinneret 15 to the point where it enters quench bath 17, i.e., the air gap, can vary and can advantageously be from about 0.5 to about 100 cm and preferably from about 1 to about 20 cm. If desired, a chimney (not shown), or shield, can be provided to isolate monofilament 16 from contact by air currents which might otherwise affect the cooling of the monofilament in some unpredictable manner. In general, barrel zone A of the extruder can be maintained at a temperature of from about 180° to 230° C., zone B at from about 190° to 230° C. and zone C at from about 190° to about 230°. Additional temperature parameters include: metering pump block 13 at from about 190° to about 230° C., spin pack 14 at from about 190° to about 230° C., spinneret 15 at from about 190° to about 230° C. and quench bath 17 at from about 30° to about 80° C.

Entering quench bath 17, monofilament 16 is passed by driven roller 18 over idler rollers 19 and 20 and thereafter is wrapped around a first godet 21 provided with nip roll 22 to prevent slippage which might otherwise result from the subsequent stretching operation. Monofilament 16 passing from godet 21 is stretched in order to effect its orientation and thereby increase its tensile strength. Techniques and conditions for drawing (i.e., stretching polypropylene monofilaments are well known to those skilled in the art. In a particularly useful embodiment, described in detail below, the polypropylene monofilament undergoes two heated draw operations.

As seen in FIG. 1 monofilament 16 is drawn through heating unit 23, which can be an oven chamber or a hot water trough, by means of second godet 24 which rotates at a higher speed than first godet 21, thereby stretching the monofilament from 4 to 7 times its original length, preferably from 6 to 7 times its original length, and more preferably from 6.5 to 6.8 times its original length. Where heating unit 23 is an oven chamber, its temperature is advantageously maintained at from about 90° to about 180° C. and preferably from about 110° to about 160° C.

Monofilament 16 is drawn a second time by passing it through heating unit 25, which can be an oven chamber or a hot water trough, by means of third godet 26. The second draw achieves a draw ratio of about 1.1 to about 1.5, preferably from about 1.3 to about 1.4. Where heating unit 25 is an oven chamber, the temperature is advantageously maintained at from about 100° C. to about 170° C., preferably, 120° C. to 150° C.

The monofilament may optionally be subjected to conditions which allow relaxation or shrinkage of the monofilament. Techniques and conditions suitable for achieving relaxation are known to those skilled in the art. A particularly useful technique is shown schematically in FIG. 1 wherein the monofilament is then passed through a third heating unit 27, e.g., maintained at a temperature of from about 100° to about 180° C. and preferably from about 110° to about 175° C., by means of a fourth godet 28 to heat-treat the monofilament prior to the equilibration and annealing operations. This third heat treatment results in on-line relaxation, or shrinkage, of the monofilament, e.g., for a recovery of from about 65 percent to about 96 percent, and preferably from about 70 percent to 76 percent, of the stretched length of the monofilament. In order to accommodate this on-line shrinkage in the monofilament, the fourth godet 28 is driven at a speed which is somewhat less than that of the third godet 26.

Following stretching and orientation and, optionally, relaxation, polypropylene monofilament from godet 28 is taken up on a spool (not shown). In preferred embodiments, the spool is then set aside for a period of time sufficient to permit the monofilament to achieve a condition of equilibration. While the period of equilibration may vary depending on the particular polypropylene resin selected and/or the conditions under which the resin is extruded, cooled and oriented, in most cases storage of the monofilament following its orientation for at least about 2 days, preferably at least about 3 days and more preferably at least about 4 days. It is generally preferred that the spooled monofilament be stored at ambient temperature, e.g., 20°-23° C., and a relative humidity of about 50%.

In carrying out the annealing operation, the desired length of equilibrated suture may be wound around a creel and the creel placed in a heating cabinet maintained at the desired temperature, e.g., 150° C., as described in U.S. Pat. No. 3,630,205. The sutures can be cut to a desired length and heat set at that desired length. As shown in U.S. Pat. No. 3,630,205, the creel may be rotated within the heating cabinet in order to insure uniform heating of the monofilament or the cabinet may be of the circulating hot air type in which case uniform heating of the monofilament will be achieved without the need to rotate the creel. Thereafter, the creel with its annealed suture is removed from the heating cabinet and when returned to room temperature, the suture is removed from the creel, conveniently by cutting the wound monofilament at opposite ends of the creel. The annealed sutures, optionally attached to surgical needles, are than ready to be packaged and sterilized.

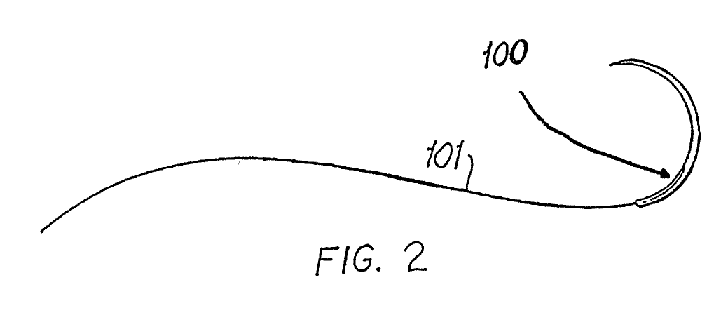

Sutures as described herein can be used to secure tissue in a desired position. suture 101, may be attached to a surgical needle 100 as shown in FIG. 2 by methods well known in the art. Wounds may be sutured by approximating tissue and passing the needled suture through tissue to create wound closure. The needle is then preferably removed from the suture and the suture tied.

The sutures and methods described herein are illustrated by the following non-limiting Example.

### COMPARATIVE EXAMPLE

Monofilament sutures ranging from size 8/0 to size 2 were fabricated from only standard polypropylene substantially in accordance with the procedure described above with respect to FIG. 1. The operating parameters and ranges are given below in Table I. Hot air ovens were used for the drawing and relaxation steps. The first draw ratio between godets 1 and 2 was 6.62. The second draw ratio between godets 2 and 3 was 1.37. The relax ratio between godets 3 and 4 was 72%.

TABLE I

| Parameter | Set Point |
|---|---|
| Pump cc/rev | 0.160-0.297 |
| Die filter | 12μ |
| Barrel 1 (° C.) | 200 ± 10 |
| Barrel 2 (° C.) | 210 ± 10 |
| Barrel 3 (° C.) | 220 ± 10 |
| Clamp (° C.) | 220 ± 10 |
| Adaptor (° C.) | 220 ± 10 |
| Block (° C.) | 220 ± 10 |
| Pump (° C.) | 220 ± 10 |
| Die (° C.) | 225 ± 15 |
| Aux die (° C.) | 225 ± 15 |
| Barrel (psi) | 1000-3000 |
| Pump (psi) | 2000 ± 500 |
| Die (psi) | 800-2000 |
| Quench (° C.) | 40 ± 10 |
| Godet 1 (meters/min, “mpm”) | 9.4 ± 0.05 |
| Godet 2 (mpm) | 62.3 ± 0.5 |
| Godet 3 (mpm) | 85.2 ± 0.5 |
| Godet 4 (mpm) | 61.3 ± 4.0 |
| Draw 1 (° C.) | 140 |
| Draw 2 (° C.) | 145 |
| Relax (° C.) | 160 ± 5 |

### EXAMPLE

Monofilament polypropylene sutures ranging from size 8/0 to size 2 were prepared in accordance with the same method as the Comparative Example except that the sutures were extruded using the conditions shown in Table II below and were made from a polypropylene polymer melt containing 0.3% by weight of PEG distearate. The polymer melt was prepared by combining a batch of standard blue polypropylene with a master batch of polypropylene containing 3.0% PEG distearate in a ratio of 9:1.

TABLE II

| Parameter | Set Point |
|---|---|
| Pump cc/rev | 0.160-0.297 |
| Die filter | 60μ |
| Barrel 1 (° C.) | 200 |
| Barrel 2 (° C.) | 200 |
| Barrel 3 (° C.) | 200 |
| Clamp (° C.) | 200 |
| Adaptor (° C.) | 200 |
| Block (° C.) | 200 |
| Pump (° C.) | 200 |
| Die (° C.) | 200 |
| Aux die (° C.) | 210 |
| Barrel (psi) | 760 |
| Pump (psi) | 500 |
| Die (psi) | 1690 |
| Quench (° C.) | 40 ± 10 |
| Godet 1 (meters/min, “mpm”) | 7.2 |
| Godet 2 (mpm) | 49.5 |
| Godet 3 (mpm) | 63.6 |
| Godet 4 (mpm) | 50.9 |
| Draw 1 (° C.) | 115 |
| Draw 2 (° C.) | 130 |
| Relax (° C.) | 153 |

The sutures of this Example modified with PEG distearate were more durable from a fray resistance point of view as compared to the sutures of the Comparative Example.

While the above description contains many specifics, these specifics should not be construed as limitations on the scope of the invention, but merely as exemplifications of preferred embodiments thereof. Those skilled in the art will envision many other possibilities within the scope and spirit of the invention as defined by the claims appended hereto.

What is claimed is:

1. A method for fabricating a polyolefin suture comprising:
a) providing a melt of at least one polyolefin, the melt containing a fatty acid diester of polyethylene glycol; and
b) extruding the melt to form a filament.

2. The method of claim 1 wherein the fatty acid ester of polyethylene glycol is a polyethylene glycol distearate.

3. The method of claim 2 wherein the percentage composition of polyethylene glycol distearate based on the total amount of polyolefin in the melt ranges from about 0.01% to about 5.0% by weight.

4. The method of claim 2 wherein the percentage composition of polyethylene glycol distearate based on the total amount of polyolefin in the melt ranges from about 0.1% to about 0.5% by weight.

5. The method of claim 2 wherein the percentage composition of polyethylene glycol distearate based on the total amount of polyolefin in the melt ranges from about 0.2% to about 0.9% by weight.

6. The method of claim 1 wherein the step of providing a melt comprises combining polyolefin with a fatty acid diester by providing a first portion of polyolefin and a second portion of polyolefin, combining the first portion of polyolefin with the fatty acid diester to form a first batch, and combining and mixing the second portion of polyolefin with the first batch to form a second batch which is heated to form the melt.

7. The method of claim 6 wherein the weight ratio of the second portion to the first batch ranges from about 2:1 to about 50:1.

8. The method of claim 6 wherein the weight ratio of the second portion to the first batch ranges from about 5:1 to about 20:1.

9. The method of claim 1 wherein the polyolefin is polypropylene.

10. The method of claim 9 wherein the polypropylene has a weight average molecular weight of from about 294,000 to about 316,000 and a number average molecular weight of from about 78,400 to about 82,000.

11. The method of claim 9 wherein the polypropylene possesses a melt flow index of from about 2 to about 6.

12. A suture fabricated in accordance with the method of claim 1.

13. A suture comprising:
a filament comprising a polyolefin and a fray reducing amount of a fatty acid diester of polyethylene glycol.

14. A suture as in claim 13 wherein the fatty acid diester is polyethylene glycol distearate.

15. A suture as in claim 13 wherein the polyolefin is polypropylene.

16. A suture as in claim 13 wherein the fatty acid diester comprises from 0.01% to 5.0% by weight of the filament.

17. A suture as in claim 13 wherein the fatty acid diester comprises from 0.2% to 0.4% by weight of the filament.

18. A suture as in claim 13 that is a monofilament suture.

19. A suture as in claim 13 wherein the polyolefin is polypropylene and the fatty acid diester is polyethylene glycol distearate which comprises about 0.2% to about 0.4% by weight of the suture.

20. A device comprising:
a needle; and
a sterilized monofilament attached to the needle, the monofilament comprising a mixture of polypropylene and 0.1% to 0.5% by weight polyethylene glycol distearate.
